(19) United States
(12) Patent Application Publication
(10) Pub. No.: US 2021/0045620 A1
(43) Pub. Date: Feb. 18, 2021

(54) OBLIQUE-VIEWING ENDOSCOPE

(71) Applicant: PANASONIC I-PRO SENSING SOLUTIONS CO., LTD., Fukuoka (JP)

(72) Inventor: Haruhiko KOHNO, Fukuoka (JP)

(73) Assignee: PANASONIC I-PRO SENSING SOLUTIONS CO., LTD., Fukuoka (JP)

(21) Appl. No.: 16/965,540

(22) PCT Filed: Sep. 25, 2019
(86) PCT No.: PCT/JP2019/037716

§ 371 Date: Jul. 28, 2020

(30) Foreign Application Priority Data
Sep. 28, 2018 (JP) 2018-185075
Dec. 17, 2018 (JP) 2018-235811

(51) Int. Cl. A61B 1/00; A61B 1/07; A61B 1/06; A61B 1/05

(52) U.S. Cl. CPC A61B 1/00179; A61B 1/07; A61B 1/051; A61B 1/00096; A61B 1/0669

(57) ABSTRACT

An oblique-viewing endoscope is equipped with a hard portion which is provided at the tip of the oblique-viewing endoscope and is formed with an oblique end surface that is inclined with respect to the axial line of the oblique-viewing endoscope so as to form an acute included angle with it; an imaging window formed in the oblique end surface; a camera which is provided in the hard portion and performs imaging through the imaging window; an illumination window which is formed in the oblique end surface at a position in the rear of the imaging window; and an illumination device which is provided in the hard portion and performs illumination through the illumination window.

### TECHNICAL FIELD

The present disclosure relates to an oblique-viewing endoscope.

### BACKGROUND ART

Oblique-viewing electronic endoscopes which are equipped with an object lens whose optical axis is inclined with respect to the endoscope axis by a prescribed inclination angle to image an observation target obliquely and an imaging device for imaging, via a prism, an optical image obtained by the object lens are known (for example, refer to PTL 1).

Oblique-viewing electronic endoscopes of this type have, in a tip surface, an oblique end surface that is inclined with respect to the endoscope axis so as to form an acute included angle with it. A lightguide for emitting light through an illumination window is disposed in the vicinity of the object lens. The lightguide is disposed on the oblique end surface on the opposite side of the object lens to the inclination side (i.e., disposed on the side where the tip portion of the endoscope projects along the endoscope axis).

### CITATION LIST

### Patent Literature

[PTL 1] JP-A-H09-122071

### SUMMARY OF INVENTION

### Technical Problem

However, in conventional oblique-viewing electronic endoscopes, a sufficiently large routing space (e.g., bending space) cannot be secured for the lightguide because the lightguide is disposed on the opposite side of the object lens to the inclination side. Where a sufficiently large bending space cannot be secured for the lightguide in oblique-viewing electronic endoscopes, the radius of curvature of its bent portion is made small, possibly resulting in a large radiation loss. This means a problem that it becomes difficult to illuminate an observation target with sufficient brightness.

The concept of the present disclosure has been conceived in view of the above circumstances in the art, and an object of the disclosure is therefore to provide an oblique-viewing endoscope in which a housing space that cover the diameter almost fully can be secured in a hard portion internal space that is located in the rear of a camera in a hard portion whose tip surface has an oblique end surface.

### Solution to Problem

The disclosure provides an oblique-viewing endoscope comprising a hard portion which is provided at the tip of the oblique-viewing endoscope and is formed with an oblique end surface that is inclined with respect to the axial line of the oblique-viewing endoscope so as to form an acute included angle with it; an imaging window formed in the oblique end surface; a camera which is provided in the hard portion and performs imaging through the imaging window; an illumination window which is formed in the oblique end surface at a position in the rear of the imaging window; and an illumination device which is provided in the hard portion and performs illumination through the illumination window.

### Advantageous Effects of Invention

The disclosure makes it possible to secure a housing space that cover the diameter almost fully in a hard portion internal space that is located in the rear of a camera in an oblique-viewing endoscope having a hard portion whose tip surface has an oblique end surface.

### BRIEF DESCRIPTION OF DRAWINGS

FIG. 1 is a perspective view showing an example of appearance of an endoscope system according to Embodiments 1 and 2.

FIG. 2 is a side view of a hard portion shown in FIG. 1.

FIG. 3 is a front view of the hard portion shown in FIG. 2.

FIG. 4 is a side sectional view taken along a first imaginary line shown in FIG. 3.

FIG. 5 is a side sectional view taken along a second imaginary line shown in FIG. 3.

FIG. 6 is a block diagram showing an example of a hardware configuration of the endoscope system.

FIG. 7 is a front view of the hard portion shown in FIG. 2.

FIG. 8 is an appearance view schematically showing the back side of a mounting circuit member.

FIG. 9 is an A-A sectional view of FIG. 5.

FIG. 10 is a block diagram showing another example of hardware configuration of the endoscope system.

### DESCRIPTION OF EMBODIMENTS

Embodiments which disclose the configuration and workings of the oblique-viewing endoscope according to the disclosure will be hereinafter described in detail by referring to the accompanying drawings when necessary. However, unnecessarily detailed descriptions may be avoided. For example, detailed descriptions of already well-known items and duplicated descriptions of constituent elements having substantially the same ones already described may be omitted. This is to prevent the following description from becoming unnecessarily redundant and thereby facilitate understanding of those skilled in the art. The following description and the accompanying drawings are provided to allow those skilled in the art to understand the disclosure sufficiently and are not intended to restrict the subject matter set forth in the claims.

### Embodiment 1

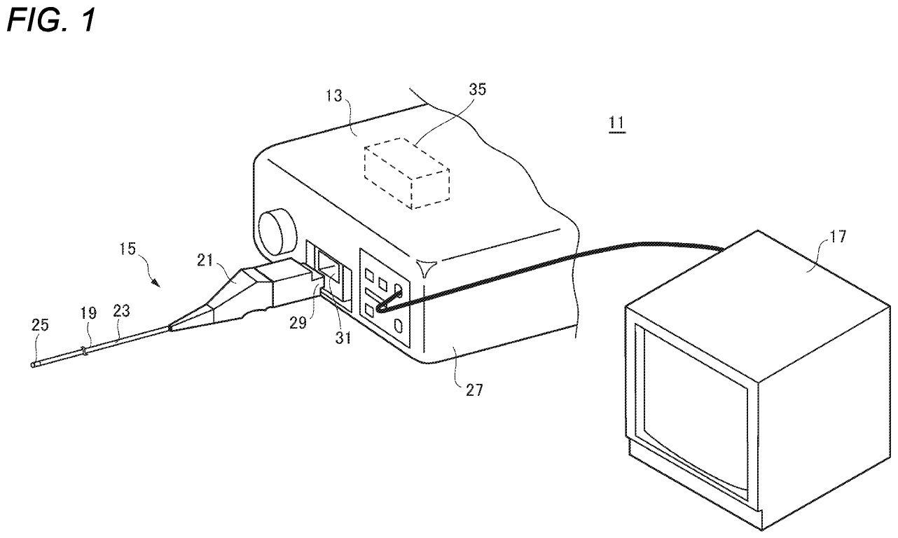

FIG. 1 is a perspective view showing an example of appearance of an endoscope system 11 according to Embodiment 1. As for terms used herein, the upward direction and the downward direction in the vertical direction of the body of a video processor 13 that is placed on a horizontal surface will be referred to as “top” and “bottom,” respectively. And the tip side (front side) of an oblique-viewing endoscope 15 will be mentioned using the term “forward” and the side on which connection is made to the video processor 13 will be mentioned using the term “rear.”

The endoscope system 11 is configured so as to include oblique-viewing endoscope 15, the video processor 13, and a monitor 17. The oblique-viewing endoscope 15 is a medical endoscope and is, for example, a hard or soft endoscope. The video processor 13 performs image processing on an image (including a still image and a moving image, for example) taken by imaging an observation target (subject; e.g., a human body or an affected part of a human body). The monitor 17 displays an image on the basis of a display signal that is output from the video processor 13. The image processing includes color correction, gradation correction, and gain adjustment, for example; however, the image processing is not limited to these kinds of processing.

The oblique-viewing endoscope 15 images an observation target. The oblique-viewing endoscope 15 is equipped with a scope 19 to be inserted into the inside of an observation target and a plug 21 to which a rear end portion of the scope 19 is connected. The scope 19 is configured so as to have a relatively long, flexible soft portion 23 and a hard portion 25 which is stiff and is provided at the tip of the soft portion 23.

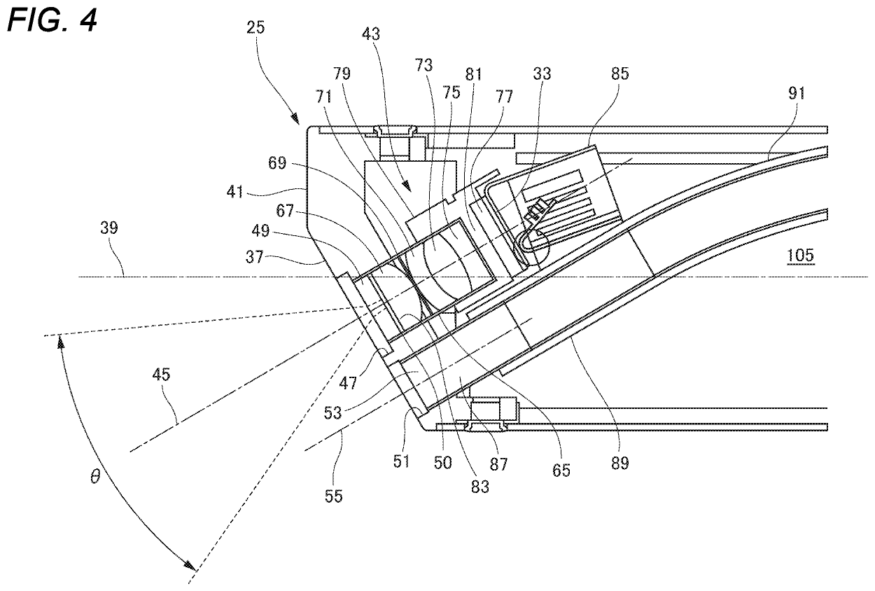

The video processor 13, which has a body 27, performs image processing on a taken image and outputs a display signal generated by the image processing. The front surface of the body 27 is provided with a socket 31 into which a base end 29 of the plug 21 is inserted. When the plug 21 is inserted into the socket 31 and the oblique-viewing endoscope 15 is thereby electrically connected to the video processor 13, power and various signals (e.g., taken image signal and control signals) can be exchanged between the oblique-viewing endoscope 15 and the video processor 13. Power and various signals are transmitted from the plug 21 to the soft portion 23 by a transmission cable (not shown) that is inserted through the scope 19. A taken image signal that is output from an image sensor 33 (see FIG. 4) provided inside the hard portion 25 is transmitted from the plug 21 to the video processor 13 via a transmission cable.

The video processor 13 performs image processing on a taken image signal that is transmitted via the transmission cable, converts image data generated by the image processing into a display signal, and outputs the display signal to the monitor 17.

The monitor 17 is a display device such as an LCD (liquid crystal display) or a CRT (cathode ray tube). The monitor 17 displays an image of a subject taken by the oblique-viewing endoscope 15. The monitor 17 displays a visible light image taken by illuminating an observation target with visible light (i.e., white light) and a fluorescence image of fluorescence light generated by illuminating an observation target with excitation light for causing emission of the fluorescence light.

An IR excitation light source 35 which is a light source of IR (infrared ray) excitation light as example excitation light is provided in the body 27 of the video processor 13. A light guide member as an illumination device is inserted in the oblique-viewing endoscope 15. When the plug 21 is inserted in the socket 31, IR excitation light emitted from the IR excitation light source 35 is transmitted to the light guide member of the oblique-viewing endoscope 15.

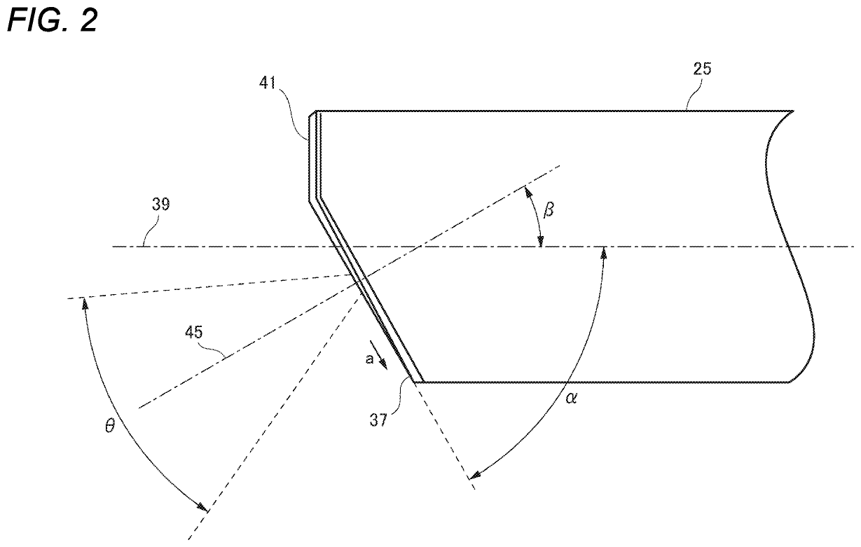

FIG. 2 is a side view of the hard portion 25 shown in FIG. 1. The hard portion 25, which is shaped like a cylinder, for example, has an oblique end surface 37 at the tip. The oblique end surface 37 is inclined with respect to the cylinder axial line 39 so as to form an acute included angle α with it. The axial line 39 is also the axial line of the oblique-viewing endoscope 15. In Embodiment 1, an orthogonal-to-axial-line end surface 41 is formed at the tip of the hard portion 25. That is, the oblique end surface 37 is a surface that is inclined rearward with respect to the orthogonal-to-axial-line end surface 41 so as to have a complementary angle (90°-α) of the included angle α. The side of the oblique end surface 37 on which it is retreated from the tip alongside the axial line 39 (more specifically, the side indicated by an arrow a in FIG. 2) will be referred to as an “inclination side.” Incidentally, the above-mentioned orthogonal-to-axial-line end surface 41 may be omitted, in which case the hard portion 25 has only the oblique end surface 37.

A camera 43 (see FIG. 4) is provided in the hard portion 25. The optical center axis 45 of the camera 43 is approximately perpendicular to the oblique end surface 37 and passes through its approximately central position. As shown in FIG. 2, for example, the view field direction of the oblique-viewing endoscope 15 may be set downward, in which case the optical center axis 45 has a dip angle β with respect to the axial line 39. The dip angle β may be set at about 30°, for example. In other words, the oblique end surface 37 is inclined rearward with respect to the orthogonal-to-axial-line end surface 41 at an angle corresponding to the dip angle β. When the oblique-viewing endoscope 15 is used actually, observation is performed by rotating it by a desired angle within 360° in a tube. Thus, when the oblique-viewing endoscope 15 is rotated by 180°, the dip angle β turns to an angle of elevation. The camera 43 has a view field of an angle θ that is centered by the optical center axis 45. In Embodiment 1, the term “view field” means an object space range where a clear image can be obtained by the optical system.

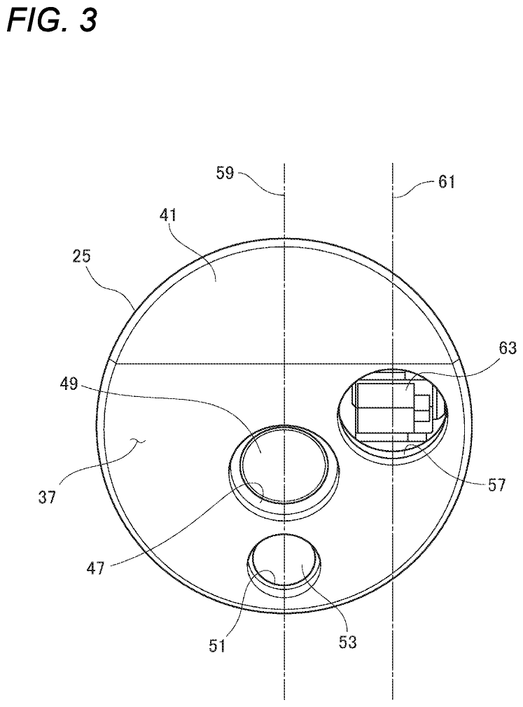

FIG. 3 is a front view of the hard portion 25 of the oblique-viewing endoscope 15 shown in FIG. 2. The oblique end surface 37 of the hard portion 25 is formed with an imaging window 47. The imaging window 47 takes in light coming from a subject. The imaging window 47 is an approximately circular (including a case of an exact circle) recess dug from the oblique end surface 37 at its approximately central position. A lens cover glass 49 is disposed at the bottom of the imaging window 47.

The oblique-viewing endoscope 15 can take a fluorescence image of fluorescence light that is emitted from a fluorescent chemical (e.g., ICG (Indocyanine Green)) administered to a subject in advance in response to illumination with excitation light (e.g., IR excitation light) for fluorescent light observation applied to an observation target region of the subject or fluorescence light that is emitted from a spontaneous fluorescent substance in a skin. In fluorescent observations, for example, spontaneous fluorescent observations employ light having a wavelength 405 nm and infrared observations employ IR excitation light in a wavelength range 690 to 820 nm. Although Embodiment 1 described below will be directed to an example of using IR excitation light as excitation light for fluorescent light observation, the excitation light is not restricted to it.

The oblique end surface 37 of the hard portion 25 is also formed with an illumination window 51. Like the imaging window 47, the illumination window 51 is a circular recess dug from the oblique end surface 37. The tip of the light guide member (e.g., optical fiber 53) which is an illumination device is disposed at the bottom of the illumination window 51. The tip of the optical fiber 53 is a light exit end surface. The base-side end, opposite to the light exit end surface, of the optical fiber 53 is connected to the plug 21. The optical fiber 53 transmits IR excitation light emitted from the IR excitation light source 35 and emits the IR excitation light through the illumination window 51. The optical fiber 53 is disposed so that an illumination optical axis 55 becomes approximately perpendicular to a portion, located on the inclination side of the optical center axis 45 of the camera 43, of the oblique end surface 37. The angles of the optical center axis 45 and the illumination optical axis 55 are not restricted to the above angles; that is, they need not always be perpendicular to the oblique end surface 37.

Furthermore, the oblique end surface 37 of the hard portion 25 is formed with an illumination window 57. Like the imaging window 47, the illumination window 57 is a circular recess dug from the oblique end surface 37. A light emitter (e.g., LED (light-emitting diode)) is disposed at the bottom of the illumination window 57 such that the illumination optical axis 55 is approximately perpendicular to the illumination window 57.

As shown in FIG. 3, the light emitter is offset and disposed on a second imaginary line 61 which is off in parallel (i.e., shifted) from a first imaginary line 59 which crosses the optical center axis 45 and the illumination optical axis 55 of the optical fiber 53. Although, in Embodiment 1, the light emitter (e.g., LED 63) emits white illumination light for illuminating a subject, there are no particular limitations on the color of illumination light.

Thus, in the oblique-viewing endoscope 15, the light guide member (e.g., optical fiber 53) which is the illumination device is connected to the IR excitation light source 35 and emits IR excitation light and the LED 63 which is the light emitter emits white illumination light.

FIG. 4 is a side sectional view taken along the first imaginary line 59 shown in FIG. 3. The hard portion 25 houses the camera 43. Having plural optical components (e.g., lenses) that form an optical path, the camera 43 introduces light coming from a subject to the optical path and forms an image. The term “light coming from a subject” as used herein includes, for example, reflection light of visible light (white light) coming from the subject and fluorescence light generated by excitation light for causing a fluorescent chemical (e.g., ICG (Indocyanine Green)) administered to the subject in advance to emit fluorescence light. The camera 43 has a cylindrical lens barrel 65 for housing the plural optical components (e.g., lenses). The outer circumference of a tip portion of the lens barrel 65 is fixed to the hard portion 25 in such a state as to be in contact with the imaging window 47.

The optical components, that is, a lens cover glass 49, a stop 50, a first lens 67, a spacer 69, a second lens 71, a third lens 73, and a fourth lens 75, are housed in the lens barrel 65 in this order from the tip side. The spacer 69, which is inserted between the first lens 67 and the second lens 71, prevents their convex surfaces from coming into contact with each other when, for example, the soft portion 23 of the oblique-viewing endoscope 15 is bent. The image sensor 33 is disposed in the rear of the fourth lens 75.

In Embodiment 1, the image sensor 33 is formed in such a manner that a solid-state imaging device (e.g., CCD (charge-coupled device) or CMOS (complementary metal-oxide-semiconductor) sensor) and a cover glass 77 disposed on the light incidence surface side of the imaging device to protect it are integrally formed.

The image sensor 33 and the lens barrel 65 are connected to each other by being fitted in a connection member 79 at the same time. As a result, the sensor center of the image sensor 33 is positioned on the optical center axis 45.

A filter-evaporated glass formed with a first excitation light cutting filter 81 by evaporation is fixed on the light incidence surface side of the image sensor 33.

In the oblique-viewing endoscope 15, a second excitation light cutting filter 83 is disposed so as to be even closer to the subject side than the first lens 67 which is disposed closest to the subject side. For example, the outer circumference of the second excitation light cutting filter 83 is fixed to the inner circumference of the lens barrel 65.

The back surface of the image sensor 33 is provided with plural pads. A flexible board 85 is electrically connected to the back surface of the image sensor 33 via the plural pads. The flexible board 85 is disposed between the image sensor 33 and a transmission cable (not shown). The flexible board 85 is formed with a transmission circuit by printing of plural-line-shaped conductor patterns. Electric wires that are bundled into the transmission cable are electrically connected to the transmission circuit by the flexible board 85. In this manner, the image sensor 33 is connected to the transmission cable by the flexible board 85. Examples of the flexible board 85 are an FFC (flexible flat cable) in which conductors that are plural band-shaped thin plates are covered with insulating sheet members to form a flexible band-shaped cable and an FPC (flexible printed wiring board) in which line-shaped conductor patterns are printed on a flexible insulating board.

The outer circumference of the optical fiber 53 which is disposed under the camera 43 is covered with a metal pipe 87. Furthermore, the outer circumference of the metal pipe 87 is covered with a resin pipe 89.

The light exit end surface of the optical fiber 53 is fixed to the illumination window 51 and the optical fiber 53 extends along the illumination optical axis 55 inward, that is, to the side opposite to the illumination window 51. The optical fiber 53 has a bent portion 91 which is bent to a direction along with the axial line 39 in a hard portion internal space that is located in the rear of the camera 43.

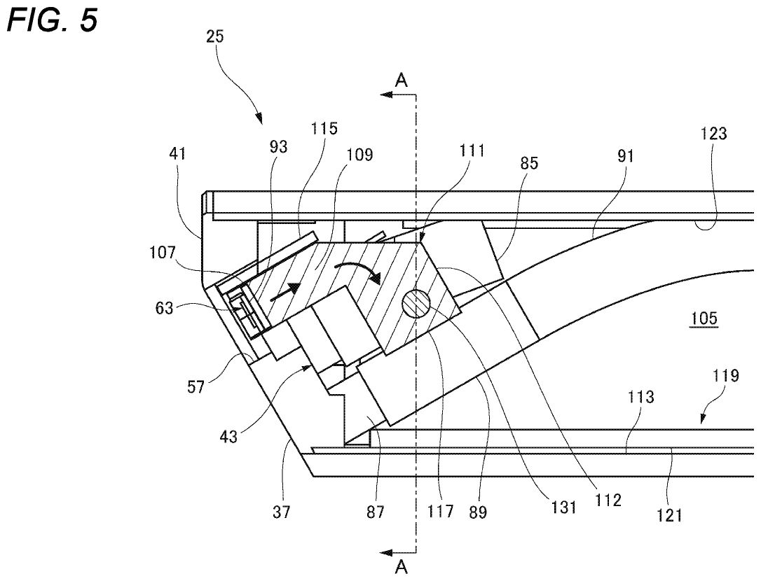

FIG. 5 is a side sectional view taken along the second imaginary line 61 shown in FIG. 3. The LED 63 which is offset from the camera 43 is disposed behind the oblique end surface 37. The light exit end surface of the LED 63 is fitted in the inner circumference of the illumination window 57 watertightly. The LED 63 is mounted on a mounting circuit member 93, for example. A transmission cable is electrically connected to the mounting circuit member 93.

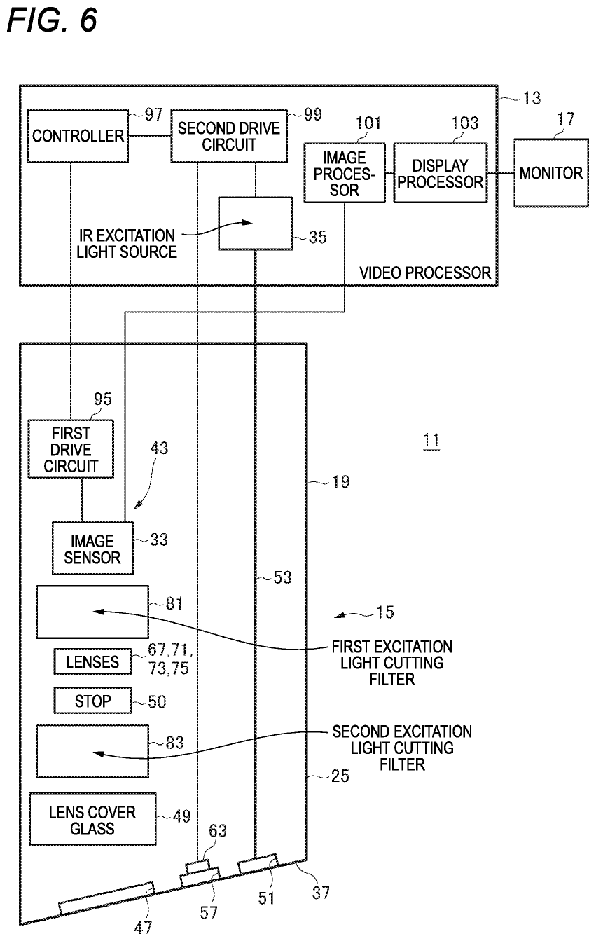

FIG. 6 is a block diagram showing an example of a hardware configuration of the endoscope system 11. The oblique-viewing endoscope 15 is equipped with a first drive circuit 95 which is formed on the flexible board 85. Operating as a drive unit, the first drive circuit 95 turns on or off an electronic shutter of the image sensor 33. When its electronic shutter is turned on by the first drive circuit 95, the image sensor 33 photoelectrically converts an optical image formed on its imaging surface and thereby outputs a taken image signal. Exposure of an optical image and generation and reading of an image signal are performed through photoelectric conversion.

The first excitation light cutting filter 81, which is disposed in front of (i.e., on the light reception side of) the image sensor 33, interrupts IR excitation light reflected from a subject and transmits visible light and fluorescence light generated by IR excitation light among light beams that pass through the lenses.

The video processor 13 is equipped with a controller 97, a second drive circuit 99, an IR excitation light source 35, an image processor 101, and a display processor 103.

The controller 97 controls a subject imaging process on the oblique-viewing endoscope 15 in a centralized manner. The controller 97 performs light emission controls on the second drive circuit 99 and performs a drive control on the first drive circuit 95 which is provided in the endoscope. The second drive circuit 99 performs a light emission control for generating IR excitation light and performs a light emission control on the LED 63.

The second drive circuit 99, which is, for example, a light source drive circuit, drives the IR excitation light source 35 and performs a light emission control during an imaging period. It is not necessary to set particular limitations on the light emission control of the IR excitation light source 35; for example, the IR excitation light source 35 may emit IR excitation light continuously, intermittently, or singly.

The term “imaging period” means a period during which the oblique-viewing endoscope 15 images an observation part. For example, the imaging period is a period from a time point when the endoscope system 11 receives a user manipulation for turning on the video processor 13 or a switch provided in the oblique-viewing endoscope 15 to a time point when the endoscope system 11 receives a turn-off user manipulation.

Having a laser diode (LD; not shown), the IR excitation light source 35 causes the LD to emit laser light (i.e., IR excitation light) in a wavelength range 690 to 820 nm to go through the optical fiber 53.

The second drive circuit 99 drives, that is, performs a light emission control on, the LED 63 and thereby causes it to emit pulse visible (i.e., white) light, for example. In an imaging period, the LED 63 illuminates a subject with pulse visible light at, for example, timing for taking a visible light image. Incidentally, in general, fluorescence light is faint. On the other hand, strong visible light is obtained even by illumination with short pulse light. It is not necessary to set particular limitations on the light emission control on the LED 63; for example, the LED 63 may emit visible light continuously, intermittently, or singly.

The second drive circuit 99 of the endoscope system 11 causes alternate output of visible light and excitation light. The endoscope system 11 is configured so that a visible light illumination timing and a timing at which to take an image of fluorescence light generated by excitation light do not overlap with each other.

The image processor 101 performs image processing on a fluorescence light image and a visible light image that are output from the image sensor 33 alternately and outputs resulting image data. Overlap between visible light and fluorescence light can be avoided by an exposure control that is performed in the image sensor 33.

The display processor 103 converts image data that is output from the image processor 101 into a display signal suitable for video display such as an NTSC (National Television System Committee) signal and outputs the display signal to the monitor 17.

The monitor 17 displays a fluorescence light image and a visible light image in, for example, the same region on the basis of a display signal that is output from the display processor 103. The monitor 17 displays the fluorescence light image and the visible light image on the same screen individually or in a superimposed manner. As a result, a user can recognize an observation target with high accuracy while looking at the fluorescence light image and the visible light image displayed on the monitor 17 individually or in a superimposed manner as a single taken image.

In the oblique-viewing endoscope 15, the illumination device may be a light emitter that emits IR excitation light. In this case, in the hard portion 25, a light emitter (e.g., LED 63) that is similar to the above-described one that emits white illumination light, is provided such that the illumination optical axis 55 is approximately perpendicular to the oblique end surface 37.

Next, the workings of the above-described oblique-viewing endoscope 15 according to Embodiment 1 will be described.

The oblique-viewing endoscope 15 according to Embodiment 1 has the hard portion 25 which is disposed at the tip of the oblique-viewing endoscope 15 and formed with the oblique end surface 37 which is inclined with respect to the axial line 39 of the oblique-viewing endoscope 15 so as to form an acute included angle with it. The oblique-viewing endoscope 15 has the imaging window 47 formed in the oblique end surface 37 and the camera 43 which is provided in the hard portion 25 and performs imaging through the imaging window 47. The oblique-viewing endoscope 15 also has the illumination window 51 which is disposed on the oblique end surface 37 at a position in the rear of the imaging window 47 and the illumination device which is provided in the hard portion 25 and performs illumination through the illumination window 51.

In the oblique-viewing endoscope 15 according to Embodiment 1, a housing space that covers the diameter almost fully can be secured in a hard portion internal space located in the rear of the camera 43.

In the oblique-viewing endoscope 15 according to Embodiment 1, the camera 43 is disposed in the direction that extends from the imaging window 47 perpendicularly to the oblique end surface 37. The illumination device is disposed in the direction that extends from the illumination window 51 perpendicularly to the oblique end surface 37.

In the oblique-viewing endoscope 15 according to Embodiment 1, a space for installation of the illumination device can be secured because the illumination device is disposed at a position in the rear of the camera 43.

In the oblique-viewing endoscope 15 according to Embodiment 1, the camera 43 is disposed in such a manner that its optical center axis 45 is approximately perpendicular to the oblique end surface 37. The illumination device is disposed in such a manner that its illumination optical axis 55 is approximately perpendicular to the oblique end surface 37. The camera 43 is disposed in such a manner that its optical center axis 45 is approximately parallel with the illumination optical axis 55 of the illumination device.

In the oblique-viewing endoscope 15 according to Embodiment 1, the illumination optical axis 55 of the illumination device is located on the oblique end surface 37 on the inclination side (i.e., the retreat side from the tip alongside the axial line 39) than the optical center axis 45 of the camera 43. In other words, the illumination device is disposed at a portion of the oblique end surface 37 which is located on the inclination side than the camera 43. The optical center axis 45 of the camera 43 and the illumination optical axis 55 of the illumination device are both approximately perpendicular to the oblique end surface 37. Thus, the optical center axis 45 and the illumination optical axis 55 are disposed approximately parallel with each other so as not to interfere with each other. Where the illumination device is a light guide member, its extension length in the direction approximately perpendicular to the oblique end surface 37 is longer than the length of the camera 43 as measured parallel with the optical center axis 45. As a result, a hard portion internal space can be secured easily in the rear of the camera 43. Where the illumination device is a light guide member, this hard portion internal space serves as a space that is effective in bending in a direction parallel to the axial line 39, the light guide member (e.g., optical fiber 53) that is inclined in a direction approximately perpendicularly to the oblique end surface 37.

As such, the oblique-viewing endoscope 15 according to Embodiment 1 can secure a housing space that covers the diameter almost fully can be secured in a hard portion internal space located in the rear of the camera 43 at the hard portion 25 having the oblique end surface 37 at the end surface.

In the oblique-viewing endoscope 15, the illumination device is a line-shaped light guide member and has the bent portion 91 which is bent along the axial line 39 in the hard portion internal space located in the rear of the camera 43.

In this oblique-viewing endoscope 15, the illumination device is a line-shaped light guide member which may be the optical fiber 53, for example. The optical fiber 53 may be either a single optical fiber 53 or, for example, a fiber bundle formed by bundling plural element optical fibers and integrating both end portions into rod-like members. The plural element optical fibers, bundled together at each tip, of the fiber bundle are fixed to each other using adhesive and each tip portion is polished to form a light exit end surface. As such, the vicinity of the tip portions of the fiber bundle becomes hard rod-like members. This light guide member has a light exit end surface that is connected to the illumination window 51 (i.e., perforation) for illumination light dug from the oblique end surface 37. In other words, the polished light exit end surface of the light guide member is parallel with the oblique end surface 37. With respect to the light guide member whose illumination optical axis 55 is approximately perpendicular to the oblique end surface 37, a portion in the vicinity of the tip of the light guide member is hard, and therefore, the portion, having a prescribed length, of the light guide member is housed in the hard portion internal space so as to extend perpendicularly to the oblique end surface 37.

For example, the hard portion 25 is circular in a front view. The first imaginary line 59 which crosses the optical center axis 45 and the illumination optical axis 55 coincides with a radial direction of the circular shape of the hard portion 25 in a plan view. The light exit end surface of the light guide member is disposed on one side of one end (i.e., the end on the inclination side) in the radial direction. Since the illumination optical axis 55 of the light guide member is approximately parallel with the optical center axis 45 and the light guide member is disposed so as not to interfere with the camera 43, the light guide member extends approximately perpendicularly to the oblique end surface 37 longer than the camera 43 extends parallel with its optical center axis 45. As a result, the inclined straight portion, in the vicinity of its tip, of the light guide member can be made long.

Since the light exit end surface of the light guide member is located at the end on the inclination side, in FIG. 4 which is a side view of the hard portion 25, the extension length of the light guide member corresponds to the length of the hypotenuse of a rectangular triangle. The height of this rectangular triangle is approximately equal to the inner diameter of the hard portion 25.

As a result, in the oblique-viewing endoscope 15, a sufficiently large routing space (bending space 105) enclosed by a rectangular triangle whose height is approximately equal to the inner diameter of the hard portion 25 can be secured on the inclination side of and in the rear of the camera 43.

The bent portion 91, having a large radius of curvature, of the light guide member for which a sufficiently large bending space 105, larger than in the conventional structure, is secured can be housed in the hard portion internal space. As a result, the light guide member can be housed in such a manner that the radiation loss of the optical waveguide due to a bend is less prone to occur.

In the oblique-viewing endoscope 15, the light emitter is disposed in the hard portion 25 in such a manner that its illumination optical axis 55 is approximately perpendicular to the oblique end surface 37.

In the oblique-viewing endoscope 15, the hard portion 25 has the light emitter (e.g., LED 63) in addition to the illumination device. As a result, the oblique-viewing endoscope 15 can incorporate different types of illumination device while taking their respective advantages. In recent years, light emitters which can emit white illumination light that is sufficiently high in light intensity have been developed. On the other hand, a light guide member (e.g., optical fiber 53) that is connected to an IR excitation light source 35 can be used to apply IR excitation light that is hard for a light emitter to give a sufficient light intensity. In the oblique-viewing endoscope 15, by using the light emitter to generate white illumination light, the cost can be made much lower than in a case that a light guide member is used for both of white illumination light and IR excitation light. Furthermore, the oblique-viewing endoscope 15 can be reduced in weight by the use of the light emitter. In many cases, a surgery is performed with the oblique-viewing endoscope 15 always held by a doctor or his or her assistant (hereinafter referred to as a “doctor or the like”). Thus, the oblique-viewing endoscope 15 which is reduced in weight can lower the burden of the doctor or the like.

In the oblique-viewing endoscope 15, the light emitter is offset and disposed on the second imaginary line 61 which is shifted in parallel from the first imaginary line 59 which crosses the optical center axis 45 and the illumination optical axis 55 of the illumination device.

In the oblique-viewing endoscope 15, by offsetting of the light emitter to the second imaginary line 61, the housing density in the diameter can be made smaller than in a case that the illumination device, the camera 43, and the light emitter are arranged on the first imaginary line 59. As a result, the hard portion 25 can be reduced in diameter in the case where the diameters of the illumination device, the camera 43, and the light emitter are fixed. Conversely, where the inner diameter of the hard portion 25 is fixed, it can load the illumination device, the camera 43, and the light emitter having larger diameters.

In the oblique-viewing endoscope 15, the illumination device is connected to the IR excitation light source 35 and emits excitation light (e.g., IR excitation light) for causing a subject to emit fluorescence light and the light emitter emits white illumination light for illuminating a subject in a wide area.

In the oblique-viewing endoscope 15, a visible light image of a subject can be obtained by illuminating the subject with white illumination light (visible light). In addition, deep blood vessel information, for example, can be obtained through fluorescence by illuminating, with IR excitation light, a fluorescent chemical that was administered to a subject before a surgery or the like. That is, both of a visible light observation and an infrared light observation are enabled.

In the oblique-viewing endoscope 15, the illumination device may be a light emitter that emits excitation light (e.g., IR excitation light) for causing a subject to emit fluorescence light and, in the hard portion 25, the illumination optical axis 55 extends approximately perpendicularly to the oblique end surface 37 and the light emitter which emits white illumination light for illuminating a subject in a wide area is provided.

In this oblique-viewing endoscope 15, the illumination device is a self-light emission member that emits IR excitation light for causing a subject to emit fluorescence light. In addition, the other light emitter which emits white illumination light for illuminating a subject in a wide area is provided. Thus, in this oblique-viewing endoscope 15, IR excitation light and white illumination light are emitted from different light emitters. In this oblique-viewing endoscope 15, since no light guide member is necessary, the cost can be made even lower than in the case where a light guide member is used. Furthermore, further weight reduction can be enabled, whereby the burden of a doctor or the like can be made even lighter. (cl Background Leading to Embodiment 2

Referential patent document 1 (described later) discloses an endoscope that is equipped with a means for effectively dissipating heat generated by a light-emitting element while producing a sufficient light quantity by the light-emitting element without increasing the diameter of an insertion portion. In this endoscope, a tip portion of a curving member of a curving portion is formed with at least one projection portion and a board to which an LED as a light emitting element is attached is fixed to each projection portion. The board is made of a material that is higher in thermal conductivity than a tip hard portion, and heat generated by the LED as a light emitting element can be conducted from the board to the curving member. Since the curving member is made of a material that is equivalent to or higher than the material of the tip hard portion in thermal conductivity, heat that has been conducted from the board to the curving member is not conducted to the tip hard portion but is further conducted to the base side of the curving member and then to a spiral pipe of a flexible pipe that is connected to the base side of the curving member.

Referential patent document 1: JP-A-2011-19570

However, in the configuration of Referential patent document 1 mentioned above, the member for conducting heat generated by the LED (light-emitting diode) from the board to the curving member is the projection portion(s) formed in the tip portion of the curving member. The projection portion is formed by forming a cut-out portion (small piece) in a thin metal plate made of SUS or the like and bending inward a base end of the cut-out portion (i.e., cut-and-erect working is performed). As a result, the projection portion may not be given a sufficient heat capacity and dissipate heat efficiently. Furthermore, since the projection portion to which the board is fixed is close to the surface of the hard portion via only an outer skin, it may be difficult to suppress the temperature of the surface of the hard portion. Still further, since the illumination optical axis of the LED is parallel with the axial line of the tip hard portion, if the size of the tip portion is made large to give it a large heat capacity and the tip portion is inclined when the same structure is applied to an oblique-viewing endoscope whose imaging direction is inclined, the volume occupied by the heat dissipation structure is increased and it becomes difficult to reduce the diameter of an insertion portion.

### Embodiment 2

In Embodiment 2, a description will be made about an example of oblique-viewing endoscope that enables efficient heat dissipation while an imaging angle of view in the oblique direction and an irradiation range overlap with each other, the temperature of the surface of a hard portion can be suppressed so as to fall within a safe range, and increase of the volume occupied by a heat dissipation structure can be suppressed.

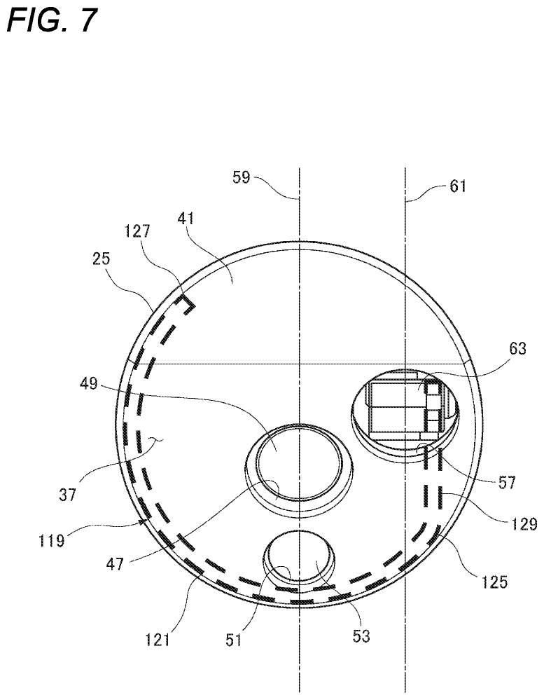

FIG. 7 is a front view of the hard portion 25 of the oblique-viewing endoscope 15 shown in FIG. 2. The oblique end surface 37 of the hard portion 25 is formed with an imaging window 47. The imaging window 47 takes in light coming from a subject. The imaging window 47 is an approximately circular (including a case of an exact circle) recess dug from the oblique end surface 37 at its approximately central position. A lens cover glass 49 is disposed at the bottom of the imaging window 47.

The oblique-viewing endoscope 15 can take a fluorescence image of fluorescence light that is emitted from a fluorescent chemical (e.g., ICG (indocyanine green)) administered to a subject in advance in response to illumination with excitation light (e.g., IR excitation light) for fluorescent observation applied to an observation target region of the subject or fluorescence light that is emitted from a spontaneous fluorescent substance in a skin. In fluorescent observations, for example, spontaneous fluorescent observations employ light having a wavelength 405 nm and infrared observations employ IR excitation light in a wavelength range 690 to 820 nm. Although Embodiment 2 described below will be directed to an example of using IR excitation light as excitation light for fluorescent light observation, the excitation light is not restricted to it.

The oblique end surface 37 of the hard portion 25 is also formed with an illumination window 51. Like the imaging window 47, the illumination window 51 is a circular recess dug from the oblique end surface 37. The tip of the light guide member (e.g., optical fiber 53) which is an illumination device is disposed at the bottom of the illumination window 51. The tip of the optical fiber 53 is a light exit end surface. The base-side end, opposite to the light exit end surface, of the optical fiber 53 is connected to the plug 21. The optical fiber 53 transmits IR excitation light emitted from the IR excitation light source 35 and emits the IR excitation light through the illumination window 51. The optical fiber 53 is disposed so that an illumination optical axis 55 becomes approximately perpendicular to a portion, located on the inclination side with respect to the optical center axis 45 of the camera 43, of the oblique end surface 37. The angles of the optical center axis 45 and the illumination optical axis 55 are not restricted to the above angles; that is, they need not always be perpendicular to the oblique end surface 37.

Furthermore, the oblique end surface 37 of the hard portion 25 is formed with an illumination window 57. Like the imaging window 47, the illumination window 57 is a circular recess dug from the oblique end surface 37. A light emitter (e.g., LED (light-emitting diode)) is disposed at the bottom of the illumination window 57 such that the illumination optical axis 55 is approximately perpendicularly to the illumination window 57.

As shown in FIG. 7, the light emitter is offset and disposed on a second imaginary line 61 which is off in parallel (i.e., shifted) from a first imaginary line 59 which crosses the optical center axis 45 and the illumination optical axis 55 of the optical fiber 53. Although, in Embodiment 2, the light emitter (e.g., LED 63) emits white illumination light for illuminating a subject, there are no particular limitations on the oscillation wavelength or wavelength range of illumination light.

Thus, in the oblique-viewing endoscope 15, the light guide member (e.g., optical fiber 53) which is the illumination device is connected to the IR excitation light source 35 and emits IR excitation light and the LED 63 which is the light emitter emits white illumination light. There are no particular limitations on the numbers of light emitters and light guide members provided in the oblique-viewing endoscope 15; no light emitter or light guide member may be provided.

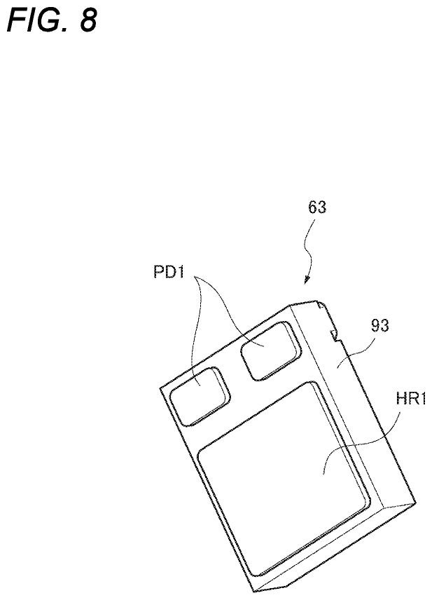

A secondary heat transmission member 119 (represented by a broken line in the figure) is provided in the hard portion 25. The secondary heat transmission member 119 is shaped like a circular arc and extends alongside the inner wall surface of the hard portion 25. The secondary heat transmission member 119 will be described later. FIG. 8 is an appearance view schematically showing the back side of a mounting circuit member 93. The LED 63 which is offset from the camera 43 is disposed behind the oblique end surface 37. The light exit end surface of the LED 63 is fitted watertightly in the inner circumference of the illumination window 57. The LED 63 is mounted on a cuboid-shaped mounting circuit member 93 (see FIG. 8) which is a board, for example. As shown in FIG. 8, the mounting circuit member 93 has, for example, two electrodes PD1 and a heat dissipation portion HR1. The light emitting element of the LED 63 is mounted on a surface that is opposite to the electrodes PD1 and the heat dissipation portion HR1 shown in FIG. 8. The two electrodes PD1 and the heat dissipation portion HR1 are arranged on the same surface and are separated and spaced from each other. In the mounting circuit member 93, tip-side lead wires (not shown) of a transmission cable are electrically connected (e.g., soldered) to the respective electrodes PD1 via the flexible board 85. The mounting circuit member 93 is made of a material (e.g., aluminum nitride) that is high in thermal conductivity. The mounting circuit member 93 transmits, efficiently, heat generated by light emission of the LED 63 to a heat receiving surface of a primary heat transmission member (described later) via the heat dissipation portion HR1 which is in contact with the primary heat transmission member. It is possible to omit the mounting circuit member 93 as shown in FIG. 8 and cause heat transfer from the heat dissipation surface of the LED 63, depending on the structure of a terminal surface on which the LED 63 is mounted.

The oblique-viewing endoscope 15 according to Embodiment 2 is disposed inside the hard portion 25 so that the primary heat transmission member 111 is housed in the hard portion 25. The primary heat transmission member 111 is disposed in the rear of the LED 63. The primary heat transmission member 111 is formed with a heat receiving surface 107. The heat receiving surface 107 is formed approximately in parallel with the oblique end surface 37 and is mounted with the LED 63, whereby heat generated by the LED 63 is transmitted to it. Thus, the primary heat transmission member 111 is made of a material (e.g., copper or aluminum) that is high in heat conductivity.

The primary heat transmission member 111 has a heat conduction portion 109 which extends from the heat receiving surface 107 in a direction that is parallel with the optical center axis 45. The heat conduction portion 109 is shaped like, for example, a rod that is quadrangle in a cross section taken perpendicularly to its extension direction. A rear end portion of the heat conduction portion 109 is formed with a connection portion 112 which is bent to a direction parallel with the heat receiving surface 107. That is, the primary heat transmission member 111 consisting of the heat conduction portion 109 and the connection portion 112 is L-shaped in a side view. A side surface of the connection portion 112 is formed with a female screw portion that is threadedly engaged with a screw 131 (described later).

A heat insulating members 115 is disposed inside the hard portion 25. The heat insulating member 115 is disposed between the primary heat transmission member 111 and a portion, closest to the primary heat transmission member 111, of the inner wall surface 113 of the hard portion 25. Another heat insulating member 115 is disposed between the primary heat transmission member 111 and the camera 43. Made of a material that is low in heat conductivity and is shaped like a sheet or a plate, the heat insulating members 115 interrupt a heat flow coming from the primary heat transmission member 111 that has received heat from the LED 63 and increased in temperature. Provided with the heat insulating members 115, the primary heat transmission member 111 can be disposed close to the inner wall surface 113 and the camera 43. This makes it easier to reduce the diameter of the hard portion 25 while suppressing temperature increase of its outer surface. The heat insulating members 115 may be replaced by air existing inside the hard portion 25. In this case, the LED 63 and the primary heat transmission member 111 are held in the air except for their portions that are in contact with the secondary heat transmission member 119.

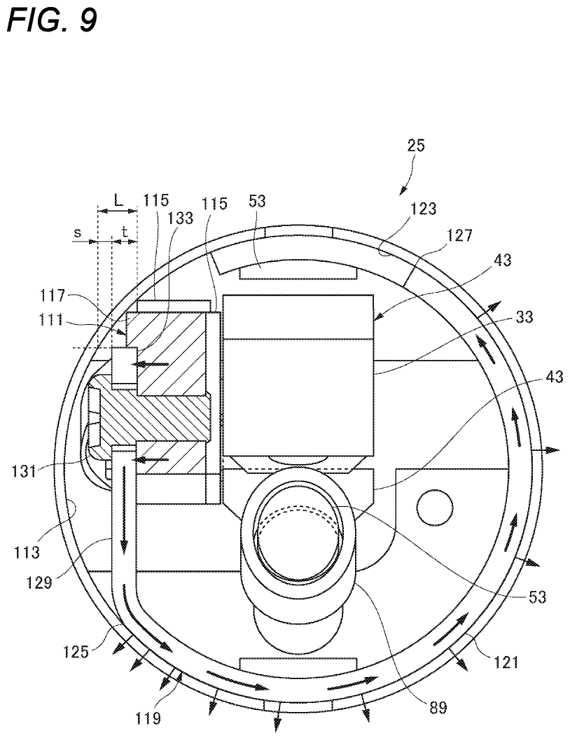

FIG. 9 is an A-A sectional view of FIG. 5. The camera 43 has a soldering surface on which plural pads are arranged like a matrix, in a back surface of the image sensor 33 which is provided at a rear position of the camera 43. Conductors of the flexible board 85 (see FIG. 4) are electrically connected to the respective pads on the soldering surface.

In the primary heat transmission member, the secondary heat transmission member 119 is connected to a tip portion 117 (in the extension direction), opposite to the heat receiving surface 107, of the heat conduction portion 109. The secondary heat transmission member 119 is shaped like a semicylinder that is curved alongside the inner wall surface 113 of the hard portion 25. The secondary heat transmission member 119 is made of a material (e.g., copper or aluminum) that is high in heat conductivity. The secondary heat transmission member 119 is integrated with or formed as a member separate from the primary heat transmission member 111. The secondary heat transmission member 119 has a heat dissipation portion 121 on the side opposite to the heat conduction portion 109. The heat dissipation portion 121 is in contact with the inner wall surface 113 of the hard portion 25.

The heat dissipation portion 121 has a length that is at least greater than or equal to approximately half of the circumferential length of the inner wall surface 113 of the hard portion 25 and is shaped like a circular arc. A portion, not in contact with the heat dissipation portion 121, of the inner wall surface 113 of the hard portion 25 is a non-contact-with-heat-dissipation-portion portion 123. The optical fiber 53 which is connected to the illumination device is disposed inside the non-contact-with-heat-dissipation-portion portion 123.

The secondary heat transmission member 119 is provided with a hanging heat transmission portion 129 between the heat conduction portion 109 and the heat dissipation portion 121. The hanging heat transmission portion 129 is disposed so as not to be in contact with the inner wall surface 113 or the other members because it extends from the tip portion 117 (in the extension direction) of the heat conduction portion 109 parallel with a radial direction of the hard portion 25. The hanging heat transmission portion 129 has a plate shape with a predetermined area. In the secondary heat transmission member 119, a heat flow coming from the heat conduction portion 109 is transmitted so as to expand two-dimensionally as it goes through the hanging heat transmission portion 129. Part of the heat that has been expanded in the hanging heat transmission portion 129 is dissipated to the internal space (filled with air, for example) in the hard portion 25 by heat convection and heat radiation. Thus, the heat dissipation portion 121, being lower in temperature than the heat conduction portion 109, of the secondary heat transmission member 119 is in contact with the inner wall surface 113.

As described above, in the hard portion 25, part of heat generated by the LED 63 is absorbed by the heat conduction portion 109 having a large heat capacity via the heat receiving surface 107 and then dissipated by the hanging heat transmission portion 129 before reaching the heat dissipation portion 121, and thus the temperature is decreased in multiple steps.

The secondary heat transmission member 119 employed in this embodiment is formed so as to be separate from the primary heat transmission member 111. A start-side end portion of the hanging heat transmission portion 129 of the secondary heat transmission member 119 is fastened to the tip portion 117 (in the extension direction) of the heat conduction portion 109 by a screw 131. The start-side end portion of the hanging heat transmission portion 129 and the tip portion 117 (in the extension direction) of the heat conduction portion 109 are in close contact with each other in a wide area to enable good heat conduction there.

A hanging heat transmission portion heat receiving surface 133, where the secondary heat transmission member 119 is connected to the primary heat transmission member 111, of the secondary heat transmission member 119 is spaced from the inner wall surface 113 of the hard portion 25 by a distance L that is longer than the thickness t of the hanging heat transmission portion 129. Thus, a gap 135 having a straight-line distance s (s=L−t) exists between a surface, opposite to the hanging heat transmission portion heat receiving surface 133, of a top portion of the hanging heat transmission portion 129 and the inner wall surface 113. The straight-line distance s of the gap 135 increases gradually as the position goes down in the hanging-down direction of the hanging heat transmission portion 129 and becomes maximum approximately at the center of the hanging heat transmission portion 129 in its hanging-down direction.

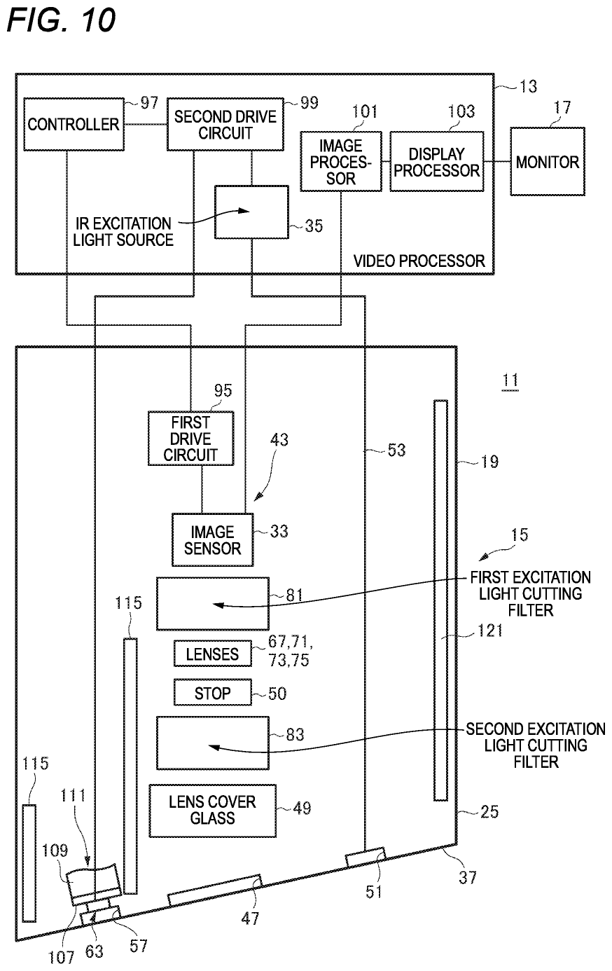

FIG. 10 is a block diagram showing an example of hardware configuration of the endoscope system 11. The oblique-viewing endoscope 15 is equipped with a first drive circuit 95 which is formed on the flexible board 85. Operating as a drive unit, the first drive circuit 95 turns on or off an electronic shutter of the image sensor 33. When its electronic shutter is turned on by the first drive circuit 95, the image sensor 33 photoelectrically converts an optical image formed on its imaging surface and thereby outputs a taken image signal. Exposure of an optical image and generation and reading of an image signal are performed through photoelectric conversion.

The first excitation light cutting filter 81, which is disposed in front of (i.e., on the light reception side of) the image sensor 33, interrupts IR excitation light reflected from a subject and transmits visible light and fluorescence light generated by IR excitation light among light beams that have passed through the lenses.

The video processor 13 is equipped with a controller 97, a second drive circuit 99, an IR excitation light source 35, an image processor 101, and a display processor 103.

The controller 97 controls a subject imaging process for the oblique-viewing endoscope 15 in a centralized manner. The controller 97 performs light emission controls on the second drive circuit 99 and performs a drive control on the first drive circuit 95 which is provided in the endoscope. The second drive circuit 99 performs a light emission control for generating IR excitation light and performs a light emission control on the LED 63.

The second drive circuit 99, which is, for example, a light source drive circuit, drives the IR excitation light source 35 and performs a light emission control during an imaging period. It is not necessary to set particular limitations on the light emission control of the IR excitation light source 35; for example, the IR excitation light source 35 may emit IR excitation light continuously, intermittently, or singly.

The term “imaging period” means a period during which the oblique-viewing endoscope 15 images an observation part. For example, the imaging period is a period from a time point when the endoscope system 11 receives a user manipulation for turning on the video processor 13 or a switch provided in the oblique-viewing endoscope 15 to a time point when the endoscope system 11 receives a turn-off user manipulation.

Having a laser diode (LD; not shown), the IR excitation light source 35 causes the LD to emit laser light (i.e., IR excitation light) in a wavelength range 690 to 820 nm to go through the optical fiber 53.

The second drive circuit 99 drives, that is, performs a light emission control on, the LED 63 and thereby causes it to emit pulse visible (i.e., white) light, for example. In an imaging period, the LED 63 illuminates a subject with pulse visible light at, for example, timing for taking a visible light image. Incidentally, in general, fluorescence light is faint. On the other hand, strong visible light is obtained by illumination of visible light even with short pulse. It is not necessary to set particular limitations on the light emission control on the LED 63; for example, the LED 63 may emit visible light continuously, intermittently, or singly.

The second drive circuit 99 of the endoscope system 11 causes alternate output of visible light and excitation light. The endoscope system 11 is configured so that a visible light illumination timing and a timing at which to take an image of fluorescence light generated by excitation light do not overlap with each other.

The image processor 101 performs image processing on a fluorescence light image and a visible light image that are output from the image sensor 33 alternately and outputs resulting image data. Overlap between visible light and fluorescence light can be avoided by an exposure control that is performed in the image sensor 33.

The display processor 103 converts image data that is output from the image processor 101 into a display signal suitable for video display such as an NTSC (National Television System Committee) signal and outputs the display signal to the monitor 17.

The monitor 17 displays a fluorescence light image and a visible light image in, for example, the same region on the basis of a display signal that is output from the display processor 103. The monitor 17 displays the fluorescence light image and the visible light image on the same screen individually or in a superimposed manner. As a result, a user can recognize an observation target with high accuracy while looking at the fluorescence light image and the visible light image displayed on the monitor 17 individually or in a superimposed manner as a single taken image.

In the oblique-viewing endoscope 15, the illumination device may be a light emitter that emits IR excitation light. In this case, in the hard portion 25, a light emitter (e.g., LED 63) that is similar to the above-described one that emits white illumination light, is provided such that the illumination optical axis 55 which is approximately perpendicular to the oblique end surface 37.

Next, the workings of the above-described oblique-viewing endoscope 15 according to Embodiment 2 will be described.

### Oblique-Viewing Structure

The oblique-viewing endoscope 15 according to Embodiment 2 has the hard portion 25 which is disposed at the tip of the oblique-viewing endoscope 15 and formed with the oblique end surface 37 which is inclined with respect to the axial line 39 of the oblique-viewing endoscope 15 so as to form an acute included angle α with it. The oblique-viewing endoscope 15 has the imaging window 47 formed in the oblique end surface 37 and the camera 43 which is provided in the hard portion 25 and performs imaging through the imaging window 47. The oblique-viewing endoscope 15 also has the illumination window 51 which is disposed on the oblique end surface 37 at a position in the rear of the imaging window 47 and the illumination device which is provided in the hard portion 25 and performs illumination through the illumination window 51.

In the oblique-viewing endoscope 15 according to Embodiment 2, a housing space that covers the diameter almost fully can be secured in a hard portion internal space located in the rear of the camera 43.

In the oblique-viewing endoscope 15 according to Embodiment 2, the camera 43 is disposed in the direction that extends from the imaging window 47 perpendicularly to the oblique end surface 37. The illumination device is disposed in the direction that extends from the illumination window 51 perpendicularly to the oblique end surface 37.

In the oblique-viewing endoscope 15 according to Embodiment 2, a space for installation of the illumination device can be secured because the illumination device is disposed at a position in the rear of the camera 43.

In the oblique-viewing endoscope 15 according to Embodiment 2, the camera 43 is disposed in such a manner that its optical center axis 45 is approximately perpendicular to the oblique end surface 37. The illumination device is disposed in such a manner that its illumination optical axis 55 is approximately perpendicular to the oblique end surface 37. The camera 43 is disposed in such a manner that its optical center axis 45 is approximately parallel with the illumination optical axis 55 of the illumination device.

In the oblique-viewing endoscope 15 according to Embodiment 2, the illumination optical axis 55 of the illumination device is located on the oblique end surface 37 on the inclination side (i.e., the retreat side from the tip alongside the axial line 39) than the optical center axis 45 of the camera 43. In other words, the illumination device is disposed at a portion of the oblique end surface 37 which is located on the inclination side than the camera 43. The optical center axis 45 of the camera 43 and the illumination optical axis 55 of the illumination device are both approximately perpendicular to the oblique end surface 37. Thus, the optical center axis 45 and the illumination optical axis 55 are disposed approximately parallel with each other so as not to interfere with each other. Where the illumination device is a light guide member, its extension length in the direction approximately perpendicular to the oblique end surface 37 is longer than the length of the camera 43 as measured parallel with the optical center axis 45. As a result, a hard portion internal space can be secured easily in the rear of the camera 43. Where the illumination device is a light guide member, this hard portion internal space serves as a space that is effective in bending, in a direction parallel with the axial line 39, the light guide member (e.g., optical fiber 53) that is inclined in a direction approximately perpendicularly to the oblique end surface 37.

As such, the oblique-viewing endoscope 15 according to Embodiment 2 can secure a housing space that covers the diameter almost fully in a hard portion internal space located in the rear of the camera 43 in the hard portion 25 having the oblique end surface 37 on the tip surface.

In the oblique-viewing endoscope 15, the illumination device is a line-shaped light guide member and has the bent portion 91 which is bent in a direction parallel with the axial line 39 in the hard portion internal space located in the rear of the camera 43.

In this oblique-viewing endoscope 15, the illumination device is a line-shaped light guide member which may be the optical fiber 53, for example. The optical fiber 53 may be either a single optical fiber 53 or, for example, a fiber bundle formed by bundling plural element optical fibers and integrating both end portions into rod-like members. The plural element optical fibers, bundled together at each tip, of the fiber bundle are fixed to each other using adhesive and each tip portion is polished to form a light exit end surface. As such, the vicinity of the tip portions of the fiber bundle becomes hard rod-like members. This light guide member has a light exit end surface that is connected to the illumination window 51 (i.e., perforation) for illumination light dug from the oblique end surface 37. In other words, the polished light exit end surface of the light guide member is parallel with the oblique end surface 37. Since the vicinity of the tip portion of the light guide member whose illumination optical axis 55 is connected approximately perpendicular to the oblique end surface 37 is hard, a portion, having a prescribed length, of the light guide member is housed in the hard portion internal space so as to extend perpendicularly to the oblique end surface 37.

For example, the hard portion 25 is circular in a front view. The first imaginary line 59 which crosses the optical center axis 45 and the illumination optical axis 55 coincides with a radial direction of the circular shape of the hard portion 25 in a plan view. The light exit end surface of the light guide member is disposed on one side of one end (i.e., the end on the inclination side) in the radial direction. Since the illumination optical axis 55 of the light guide member is approximately parallel with the optical center axis 45 and the light guide member is disposed so as not to interfere with the camera 43, the light guide member extends approximately perpendicularly to the oblique end surface 37 longer than the camera 43 extends parallel with its optical center axis 45. As a result, the inclined straight portion, in the vicinity of its tip, of the light guide member can be made long.

Since the light exit end surface of the light guide member is located at the end on the inclination side, in FIG. 4 which is a side view of the hard portion 25, the extension length of the light guide member corresponds to the length of the hypotenuse of a rectangular triangle. The height of this rectangular triangle is approximately equal to the inner diameter of the hard portion 25.

As a result, in the oblique-viewing endoscope 15, a sufficiently large routing space (bending space 105) enclosed by a rectangular triangle whose height is approximately equal to the inner diameter of the hard portion 25 can be secured on the inclination side of and in the rear of the camera 43.

The bent portion 91, having a large radius of curvature, of the light guide member for which a sufficiently large bending space 105, larger than in the conventional structure, is secured can be housed in the hard portion internal space. As a result, the light guide member can be housed in such a manner that the radiation loss of the optical waveguide due to a bend is less prone to occur.

In the oblique-viewing endoscope 15, the light emitter is disposed in the hard portion 25 in such a manner that its illumination optical axis 55 is approximately perpendicular to the oblique end surface 37.

In the oblique-viewing endoscope 15, the hard portion 25 has the light emitter (e.g., LED 63) in addition to the illumination device. As a result, the oblique-viewing endoscope 15 can incorporate different types of illumination device while taking their respective advantages. In recent years, light emitters which can emit white illumination light that is sufficiently high in light intensity have been developed. On the other hand, a light guide member (e.g., optical fiber 53) that is connected to an IR excitation light source 35 can be used to apply IR excitation light that is hard for a light emitter to give a sufficient light intensity. In the oblique-viewing endoscope 15, by using the light emitter to generate white illumination light, the cost can be made much lower than in a case that a light guide member is used for both of white illumination light and IR excitation light. Furthermore, the oblique-viewing endoscope 15 can be reduced in weight by the use of the light emitter. In many cases, a surgery is performed with the oblique-viewing endoscope 15 always held by a doctor or his or her assistant (hereinafter referred to as a “doctor or the like”). Thus, the oblique-viewing endoscope 15 which is reduced in weight can lower the burden of the doctor or the like.

In the oblique-viewing endoscope 15, the illumination device is connected to the IR excitation light source 35 and emits excitation light (e.g., IR excitation light) for causing a subject to emit fluorescence light and the light emitter emits white illumination light for illuminating a subject in a wide area.

In the oblique-viewing endoscope 15, a visible light image of a subject can be obtained by illuminating the subject with white illumination light (visible light). In addition, deep blood vessel information, for example, can be obtained through fluorescence by illuminating, with IR excitation light, a fluorescent chemical that was administered to a subject before a surgery or the like. That is, both of a visible light observation and an infrared light observation are enabled.

In the oblique-viewing endoscope 15, the illumination device may be a light emitter that emits excitation light (e.g., IR excitation light) for causing a subject to emit fluorescence light and, in the hard portion 25, the illumination optical axis 55 extends approximately perpendicularly to the oblique end surface 37 and the light emitter which emits white illumination light for illuminating a subject in a wide area is provided.

In this oblique-viewing endoscope 15, the illumination device is a self-light emission member that emits IR excitation light for causing a subject to emit fluorescence light. In addition, the other light emitter which emits white illumination light for illuminating a subject in a wide area is provided. Thus, in this oblique-viewing endoscope 15, IR excitation light and white illumination light are emitted from different light emitters. In this oblique-viewing endoscope 15, since no light guide member is necessary, the cost can be made even lower than in the case where a light guide member is used. Furthermore, further weight reduction can be enabled, whereby the burden of a doctor or the like can be made even lighter.

### Heat Dissipation Structure

The oblique-viewing endoscope 15 according to Embodiment 2 has the hard portion 25 which is provided at the tip of the scope 19 and is formed with the oblique end surface 37 which is inclined with respect to the tip-side axial line 39 of the scope 19 so as to form the acute included angle a with it. The oblique-viewing endoscope 15 has the camera 43 which is disposed inside the hard portion 25 and whose optical center axis 45 is approximately perpendicular to the oblique end surface 37 and the light emitter (e.g., LED 63) which is disposed inside the hard portion 25 and emits illumination light from the oblique end surface 37. The oblique-viewing endoscope 15 has the heat receiving surface 107 which is formed approximately parallel with the oblique end surface 37, is mounted with the LED 63, and receives heat generated by the LED 63, and also has the primary heat transmission member 111 having the heat conduction portion 109 which extends from the heat receiving surface 107 in the direction that is parallel with the optical center axis 45.

In the oblique-viewing endoscope 15, the LED 63 is fixed to the heat receiving surface 107 of the heat conduction portion 109 of the primary heat transmission member 111. The primary heat transmission member 111 is a solid block body made of a metal that is high in thermal conductivity, such as copper or aluminum. The heat conduction portion 109 is shaped like a rod and extends in the direction that is parallel with the optical center axis 45 of the camera 43 which is approximately perpendicular to the oblique end surface 37. Since the board of the LED 63 is fixed, in parallel, to the heat receiving surface 107 of one end surface of the heat conduction portion 109 which is shaped like a rod, its illumination range can overlap with the imaging angle of view of the camera 43 which is directed obliquely.

A cross section, taken perpendicularly to its extension direction, of the heat conduction portion 109 has approximately the same area as the heat receiving surface 107. The heat conduction portion 109 is a block body that has the sectional area approximately the same as the area of the heat receiving surface 107 and extends in the direction that is parallel with the optical center axis 45. On the other hand, in the conventional heat dissipation structure, the projection portion(s) to which the board of the LED 63 is fixed is formed by forming a cut-out portion (small piece) in a thin metal plate made of SUS or the like and bending a base end of the cut-out portion. In this heat dissipation structure, the cross section that contributes to heat conduction is the cross section of the base end (bent portion) of the cut-out portion (small piece). Thus, the cross section, contributing to heat conduction, of the heat conduction portion 109 of the primary heat transmission member 111 is much larger than that of the projection portion. Furthermore, the heat capacity, which relates to the product of the specific weight and the specific heat of an object, can be made sufficiently larger than that of the projection portion. In other words, the thermal resistance, which is a concept based on the analogy between heat and electricity, of the heat conduction portion 109 of the primary heat transmission member 111 is sufficiently smaller than that of the projection portion. As a result, in the heat dissipation structure in which the LED 63 is mounted on the heat receiving surface 107 which is formed in the heat conduction portion 109 of the primary heat transmission member 111, a more efficient heat flow can be obtained than in the heat dissipation structure in which the LED 63 is mounted on the projection portion. As a result, the heat dissipation efficiency can be increased to a large extent.

In the primary heat transmission member 111, no portion in the vicinity of the portion connected to the LED 63 comes into contact with the inner wall surface 113 of the hard portion 25 because the heat conduction portion 109 extends from the heat receiving surface 107 in the direction that is parallel with the optical center axis 45. Thus, a phenomenon that a flow of heat generated by the LED 63 is transmitted to the hard portion 25 by short-distance heat conduction and increases the temperature of the surface of the hard portion 25 can be suppressed.

Furthermore, the heat conduction portion 109 is disposed so as to extend in the direction that is parallel with the optical center axis 45. Thus, in the internal space of the cylindrical hard portion 25, the heat conduction portion 109 can be disposed from one end to the other end of a diameter in the direction inclined with respect to the axial line 39, utilizing the wide housing space. In other words, the housing structure has a margin by virtue of the shape and posture/orientation that enable effective use of the wide housing space. In the heat dissipation structure of the oblique-viewing endoscope 15, the diameter of the hard portion 25 can be reduced accordingly.

As a result, in the oblique-viewing endoscope 15 according to Embodiment 2, efficient heat dissipation is enabled while the imaging angle of view in the oblique viewing direction and the illumination range overlap with each other, the temperature of the surface of the hard portion can be suppressed so as to fall within a safe range, and increase of the volume occupied by the heat dissipation structure can be suppressed.

Furthermore, in the oblique-viewing endoscope 15 according to Embodiment 2, the heat insulating members 115 are disposed between the primary heat transmission member 111 and a portion, closest to the primary heat transmission member 111, of the inner wall surface 113 of the hard portion 25 and between the primary heat transmission member 111 and the camera 43.

In the oblique-viewing endoscope 15, the temperature of the primary heat transmission member 111 is increased when heat is transmitted from the LED 63. The temperature-increased primary heat transmission member 111 is spaced from the inner wall surface 113 of the hard portion 25 and the camera 43 via air layers. Where these separation distances are short, heat is transmitted by heat convection and heat radiation. Such a heat movement phenomenon between a solid and a fluid that involves heat conduction, heat convection, and heat radiation is called heat transfer. The heat insulating members 115 are disposed between the primary heat transmission member 111 and a portion, closest to the primary heat transmission member 111, of the inner wall surface 113 of the hard portion 25 and between the primary heat transmission member 111 and the camera 43. The heat insulating members 115 can suppress heat transfer from the primary heat transmission member 111 effectively because it is formed by laying a foil or the like having high reflectance on a material that is low in thermal conductivity. In this manner, the heat dissipation structure in which the heat insulating members 115 are attached to the primary heat transmission member 111 can suppress the temperatures of the camera 43 and the surface of the hard portion to within safe ranges.

Incidentally, the surface, opposite to the surface in contact with the primary heat transmission member 111, of the heat insulating member 115 may be in contact with the inner wall surface 113 of the hard portion 25. In this case, the heat insulating member 115 also serves as a support member for supporting the primary heat transmission member 111 with the inner wall surface 113.

In the oblique-viewing endoscope 15 according to Embodiment 2, the secondary heat transmission member 119 is connected to the tip portion 117 (in the extension direction), opposite to the heat receiving surface 107, of the heat conduction portion 109 so as to be integrated with or be a member separate from the tip portion 117. The secondary heat transmission member 119 has, on the side opposite to the heat conduction portion 109, the heat dissipation portion 121 which is in contact with the inner wall surface 113 of the hard portion 25.

In the oblique-viewing endoscope 15, the secondary heat transmission member 119 is connected to the tip portion 117 (in the extension direction) of the heat conduction portion 109. Thus, heat generated by the LED 63 and transmitted to the heat receiving surface 107 passes through the heat conduction portion 109 having a large heat capacity and then flows to the secondary heat transmission member 119 which is connected to the tip portion 117 (in the extension direction). Part of the heat transmitted to the heat conduction portion 109 is dissipated to the internal space of the hard portion 25 from the surfaces of the heat conduction portion 109. As a result, the temperature transmitted to the secondary heat transmission member 119 is lower than that of the heat receiving surface 107. The heat that has been transmitted to the secondary heat transmission member 119 is transmitted to the hard portion 25 from the heat dissipation portion 121 which is in contact with the inner wall surface 113 of the hard portion 25. The heat that has been transmitted to the hard portion 25 is finally dissipated to the outside from the surface of the hard portion 25 that is in a safe temperature range.

In the oblique-viewing endoscope 15 according to Embodiment 2, the illumination device is disposed inside the hard portion 25 in such a manner that the illumination optical axis 55 is approximately perpendicular to the oblique end surface 37. The LED 63 is offset and disposed on the second imaginary line 61 which is shifted in parallel from the first imaginary line 59 which crosses the optical center axis 45 and the illumination optical axis 55.

In the oblique-viewing endoscope 15, because of the offsetting of the LED 63 to the second imaginary line 61, the housing density in the diameter direction can be made smaller than in a case that the illumination device, the camera 43, and the LED 63 are arranged on the first imaginary line. As a result, the hard portion 25 can be reduced in diameter in the case where the diameters of the illumination device, the camera 43, and the LED 63 are fixed. Conversely, where the inner diameter of the hard portion 25 is fixed, it can load the illumination device, the camera 43, and the LED 63 member having larger diameters.

In the oblique-viewing endoscope 15 according to Embodiment 2, the heat dissipation portion 121 has a length that is at least greater than or equal to approximately half of the circumferential length of the inner wall surface 113 of the hard portion 25 and is shaped like a circular arc. The optical fiber 53 which is connected to the illumination device is disposed inside the non-contact-with-heat-dissipation-portion portion 123, that is, the portion, not in contact with the heat dissipation portion 121, of the inner wall surface 113.

In the oblique-viewing endo scope 15, the heat dissipation portion 121 of the secondary heat transmission member 119 formed along the circumferential direction of the inner wall surface 113 of the hard portion 25 and is shaped like a circular arc. The circular arc has a length that is at least greater than or equal to approximately half of the circumferential length. Since the heat dissipation portion 121 is in contact with the cylindrical inner wall surface 113 of the hard portion 25 over the length of an arc that is at least greater than or equal to approximately half of the circumferential length of the inner wall surface 113, a large area of contact with the inner wall surface 113 can be secured without interference with the other members housed inside the inner wall surface 113. As a result, efficient heat dissipation can be performed without inhibition of reducing the diameter. The circular-arc-shaped heat dissipation portion 121 is high in temperature at a contact start position 125 where contact with the inner wall surface 113 is started and the temperature of a contact end position 127 of the heat dissipation portion 121 which extends in the circumferential direction being kept in contact with the inner wall surface 113 decreases from the temperature at the contact start position 125. This is due to heat dissipation from the inner wall surface 113 to the outside. Because of a small temperature difference at the contact end position 127, the degree of heat conduction (and hence the heat dissipation rate) is low there. On the other hand, the inner wall surface 113 of the hard portion 25 which is in contact with the circular-arc-shaped heat dissipation portion 121 has the non-contact-with-heat-dissipation-portion portion 123, that is, the portion not in contact with the heat dissipation portion 121, in the circumferential direction. The optical fiber 53 is disposed inside the non-contact-with-heat-dissipation-portion portion 123 at a position close to the inner wall surface 113 and extends in a direction that is parallel with the axial line 39 of the hard portion 25. In the heat dissipation structure of the oblique-viewing endoscope 15, the light guide member (optical fiber 53) of the illumination device which is provided separately from the LED 63 is disposed by effectively utilizing the space (i.e., the non-contact-with-heat-dissipation-portion portion 123) where the heat dissipation effect of the heat dissipation portion 121 is low.

In the oblique-viewing endoscope 15 according to Embodiment 2, the secondary heat transmission member 119 is provided with, between the heat conduction portion 109 and the heat dissipation portion 121, the hanging heat transmission portion 129 which is not in contact with the other members.

In the oblique-viewing endoscope 15, the secondary heat transmission member 119 is provided with the hanging heat transmission portion 129. The hanging heat transmission portion 129 is disposed between the tip portion 117 (in the extension direction) of the heat conduction portion 109 of the primary heat transmission member 111 and the heat dissipation portion 121 of the secondary heat transmission member 119. That is, the portion, between the tip portion 117 (in the extension direction) of the primary heat transmission member 111 and the heat dissipation portion 121, of the secondary heat transmission member 119 is exposed to the internal space of the hard portion 25 without being in contact with the other members. The internal space of the hard portion 25 is filled with air. As a result, heat is dissipated to the air by heat convection and heat radiation. The heat transfer rates of these heat convection and heat radiation are lower than the heat transfer rate of heat conduction. Thus, the hanging heat transmission portion 129 functions like a capacitor as is understood from the analogy between heat and electricity. Thus, the temperature of the hanging heat transmission portion 129 to which heat flows from the primary heat transmission member 111 is increased to a prescribed temperature and heat can be transmitted to the heat dissipation portion 121 efficiently by a temperature difference (gradient) occurring between the hanging heat transmission portion 129 and the heat dissipation portion 121.

Incidentally, the temperature of a portion of the outer surface at the contact start position 125 where the heat dissipation structure comes into contact with the inner wall surface 113 first is lowered to a safe range because of the heat capacity of the heat conduction portion 109 of the primary heat transmission member 111 and heat absorption by heat convection and heat radiation from the hanging heat transmission portion 129 of the secondary heat transmission member 119.

In the oblique-viewing endoscope 15 according to Embodiment 2, the hanging heat transmission portion heat receiving surface 133, connected to the primary heat transmission member 111, of the secondary heat transmission member 119 is spaced from the inner wall surface 113 of the hard portion 25 by the distance L which is greater than the thickness t of the hanging heat transmission portion 129.

In the oblique-viewing endoscope 15, the surface, opposite to the hanging heat transmission portion heat receiving surface 133, of a top end portion of the hanging heat transmission portion 129 has the gap 135 that is distant from the inner wall surface 113 by straight-line distance s (s=L−t). The gap 135 functions as a thermal resistance when heat is transmitted from the surface opposite to the hanging heat transmission portion heat receiving surface 133 to the inner wall surface 113. The straight-line distance s is set so that this heat resistance is at least larger than a heat resistance that is obtained when the hanging heat transmission portion 129 is brought into close contact with the inner wall surface 113 via a heat insulating member 115 as mentioned above. In this manner, in the hard portion 25, increase of the number of components is suppressed by omitting a heat insulating member 115 by securing the gap 135.

In the oblique-viewing endoscope 15 according to Embodiment 2, the primary heat transmission member 111 and the secondary heat transmission member 119 are formed as separate members and the secondary heat transmission member 119 is fastened to the tip portion 117 (in the extension direction) of the heat conduction portion 109 by the screw 131.

In the oblique-viewing endoscope 15, the primary heat transmission member 111 and the secondary heat transmission member 119 are formed as separate members. The primary heat transmission member 111 which is desired to have a large heat capacity can be made of, for example, copper which has a large specific heat. The secondary heat transmission member 119 which is desired to have good contact with the inner wall surface 113 can be made of, for example, aluminum. Even where the primary heat transmission member 111 and the secondary heat transmission member 119 are made of such different kinds of metals, they can be connected easily using the screw 131. Since the primary heat transmission member 111 and the secondary heat transmission member 119 which are separate members can be attached to each other, good performance of housing these members in the internal space of the hard portion 25 which is very small can be obtained, whereby the productivity can be increased.

The oblique-viewing endoscope 15 according to Embodiment 2 further has the mounting circuit member 93 which is mounted with the LED 63 and has the two electrodes PD1 to which the tips of the transmission cable extending from the base side are connected and the heat dissipation portion HR1 for releasing heat generated by light emission of the LED 63 to the primary heat transmission member 111. The electrodes PD1 and the heat dissipation portion HR1 are spaced from each other.

In the mounting circuit member 93 of the oblique-viewing endoscope 15, the two electrodes PD1 to which the tips of the transmission cable are connected and the heat dissipation portion HR1 are separated and spaced from each other. As a result, heat generated by light emission of the LED 63 is transmitted efficiently to the primary heat transmission member 111 via the heat dissipation portion HR1 in a state that it is spaced from the electrodes PD1. And the lead wires of the transmission cable can be connected to the respective electrodes PD1 simply, which enables efficient manufacture.

The present disclosure includes various oblique-viewing endoscopes described below:

A first oblique-viewing endoscope comprises:

a hard portion which is provided at the tip of a scope and is formed with an oblique end surface that is inclined with respect to the axial line of the scope so as to form an included angle with it;

a camera which is provided inside the hard portion in such a manner that its optical center axis is approximately perpendicular to the oblique end surface;

a light emitter which is provided inside the hard portion and emits illumination light from the oblique end surface; and

a primary heat transmission member which has a heat receiving surface that receives heat generated by and coming from the light emitter and has a heat conduction portion that extends from the heat receiving surface in a direction that is parallel with the optical center axis.

In a second oblique-viewing endoscope, which is based on the above first oblique-viewing endoscope, a heat insulating member is provided between the primary heat transmission member and a portion, closest to the primary heat transmission member, of an inner wall surface of the hard portion and between the primary heat transmission member and the camera.

In a third oblique-viewing endoscope, which is based on the above first or second oblique-viewing endoscope,

a secondary heat transmission member is connected to an extension direction tip portion, opposite to the heat receiving surface, of the heat conduction portion so as to be integrated with or formed as a member separate from the heat conduction portion;

the secondary heat transmission member has a heat dissipation portion on the side opposite to the heat conduction portion; and

the heat dissipation portion is in contact with an inner wall surface of the hard portion.

In a fourth oblique-viewing endoscope, which is based on the above third oblique-viewing endoscope,

an illumination device is provided inside the hard portion in such a manner that its illumination optical axis is approximately perpendicular to the oblique end surface; and

the light emitter is offset and disposed on a second imaginary line that is shifted in parallel from a first imaginary line that crosses the optical center axis and the illumination optical axis.

In a fifth oblique-viewing endoscope, which is based on the above fourth oblique-viewing endoscope,

the heat dissipation portion is shaped like a circular arc and has a length that is at least greater than or equal to approximately half of a circumferential length of an inner wall surface of the hard portion; and

an optical fiber that is connected to the illumination device is provided in a non-contact-with-heat-dissipation-portion portion, not in contact with the heat dissipation portion, of the inner wall surface.

In a sixth oblique-viewing endoscope, which is based on any of the above third to fifth oblique-viewing endoscopes, the secondary heat transmission member is provided with a hanging heat transmission portion that is not in contact with other members between the heat conduction portion and the heat dissipation portion.

In a seventh oblique-viewing endoscope, which is based on the above sixth oblique-viewing endoscope, a hanging heat transmission portion heat receiving surface of the secondary heat transmission member, connected to the primary heat transmission member is spaced from an inner wall surface of the hard portion by a distance that is greater than the thickness of the hanging heat transmission portion.

In an eighth oblique-viewing endoscope, which is based on any of the above third to seventh oblique-viewing endoscopes,

the primary heat transmission member and the secondary heat transmission member are formed as separate members; and

the secondary heat transmission member is fastened to the extension direction tip portion of the heat conduction portion by a screw.

In a ninth oblique-viewing endoscope, which is based on any of the above first to eighth oblique-viewing endoscopes,

a mounting circuit member which is mounted with the light emitter and has an electrode to which the tip of a transmission cable extending from the base side is connected and a heat dissipation portion which dissipates heat generated by light emission of the light emitter to the primary heat transmission member is further provided; and

the electrode and the heat dissipation portion are spaced from each other.

Although the embodiments have been described above with reference to the drawings, it goes without saying that the disclosure is not limited to those examples. It is apparent that those skilled in the art would conceive various changes, modifications, replacements, additions, deletions, or equivalents within the confines of the claims, and they are construed as being included in the technical scope of the disclosure. Constituent elements of the above-described embodiments can be combined without departing from the gist of the invention.

The present application is based on Japanese Patent Application No. 2018-185075 filed on Sep. 28, 2018 and No. 2018-235811 filed on Dec. 17, 2018, the disclosures of which are invoked in this application by reference.

### INDUSTRIAL APPLICABILITY

The present application is useful in providing oblique-viewing endoscopes in which a housing space covers the diameter almost fully can be secured in a hard portion internal space located in the rear of a camera in a hard portion whose tip surface has an oblique end surface.

### REFERENCE SIGNS LIST

15: Oblique-viewing endoscope

19: Scope

25: Hard portion

35: IR excitation light source

37: Oblique end surface

39: Axial line

43: Camera

45: Optical center axis

47: Imaging window

53: Optical fiber

55: Illumination optical axis

57: Illumination window

59: First imaginary line

61: Second imaginary line

63: LED (self light emitting member)

91: Bent portion

1. An oblique-viewing endoscope comprising:
a hard portion which is provided at a tip of the oblique-viewing endoscope and is formed with an oblique end surface that is inclined with respect to an axial line of the oblique-viewing endoscope so as to form an acute angle with it;
an imaging window formed in the oblique end surface;
a camera which is provided in the hard portion and images through the imaging window;
an illumination window which is provided in the oblique end surface at a position in the rear of the imaging window; and
an illumination device which is provided in the hard portion and illuminates through the illumination window.

2. The oblique-viewing endoscope according to claim 1,
wherein the camera is disposed at a position that is distant from the imaging window in a direction perpendicular to the oblique end surface.

3. The oblique-viewing endoscope according to claim 1,
wherein the camera is disposed in such a manner that its optical center axis is approximately perpendicular to the oblique end surface.

4. The oblique-viewing endoscope according to claim 1,
wherein the illumination device is disposed at a position that is distant from the illumination window in a direction perpendicular to the oblique end surface.

5. The oblique-viewing endoscope according to claim 1,
wherein the illumination device is disposed in such a manner that its illumination optical axis is approximately perpendicular to the oblique end surface.

6. The oblique-viewing endoscope according to claim 1,
wherein the camera is disposed in such a manner that its optical center axis is approximately parallel with the illumination optical axis of the illumination device.

7. The oblique-viewing endoscope according to claim 1,
wherein the illumination device is a line-shaped light guide member and has a bent portion that is bent in a direction along the axial line in a hard portion internal space located in the rear of the camera.

8. The oblique-viewing endoscope according to claim 1,
wherein a light emitter is provided in the hard portion in such a manner that its illumination optical axis extends approximately perpendicularly to the oblique end surface.

9. The oblique-viewing endoscope according to claim 8,
wherein the light emitter is offset and disposed on a second imaginary line that is shifted in parallel from a first imaginary line that crosses the optical center axis of the camera and the illumination optical axis of the illumination device.

10. The oblique-viewing endoscope according to claim 8,
wherein the illumination device is connected to an excitation light source and emits excitation light for causing a subject to emit fluorescence light; and
the light emitter emits white illumination light for illuminating the subject.

11. The oblique-viewing endoscope according to claim 1,
wherein the illumination device is a light emitter that emits excitation light for causing a subject to emit fluorescence light; and
a light emitter whose illumination optical axis is approximately perpendicular to the oblique end surface and that emits white illumination light for illuminating the subject is provided in the hard portion.
